(19) United States
(12) Patent Application Publication
(10) Pub. No.: US 2026/0355260 A1
(43) Pub. Date: Nov. 12, 2026

(54) RECOMBINANT EXPRESSION VECTOR FOR CONANTOKIN-G SECRETION AND ATTENUATED SALMONELLA STRAIN TRANSFORMED THEREWITH

(71) Applicant: THE INDUSTRY & ACADEMIC COOPERATION IN CHUNGNAM NATIONAL UNIVERSITY (IAC), Daejeon (KR)

(72) Inventors: Hyo Jin LEE, Daejeon (KR); Heung Jin JEON, Daejeon (KR); Solbi KIM, Daejeon (KR); Eunji KIM, Daejeon (KR); Minju HAN, Chungcheongbuk-do (KR); Myung-won LEE, Sejong (KR); Sora KANG, Daejeon (KR)

(73) Assignee: THE INDUSTRY & ACADEMIC COOPERATION IN CHUNGNAM NATIONAL UNIVERSITY (IAC), Daejeon (KR)

(21) Appl. No.: 18/997,453

(22) PCT Filed: Jul. 6, 2023
(86) PCT No.: PCT/KR2023/009607

§ 371 Date: Jan. 21, 2025

(30) Foreign Application Priority Data
Jul. 21, 2022 (KR) 10-2022-0090612

(51) Int. Cl. A61K 35/74; A61K 38/00; A61P 35/00; C07K 7/08; C07K 14/195; C12N 1/20; C12N 15/74; C12R 1/42

(52) U.S. Cl. CPC A61K 35/74; A61P 35/00; C07K 7/08; C07K 14/195; C12N 1/20; C12N 15/74; A61K 38/00; C07K 2319/00; C12R 2001/42

(57) ABSTRACT

The present disclosure relates to a recombinant expression vector for secretion of conantokin-G and an attenuated Salmonella strain transformed therewith, and provides: an attenuated Salmonella strain transformed with a recombinant expression vector including a flgM gene and a conantokin-G gene; an anticancer pharmaceutical composition including the attenuated Salmonella strain as an active ingredient; and a recombinant expression vector capable of enhancing secretion capacity for conantokin-G.

### TECHNICAL FIELD

The present disclosure relates to a recombinant expression vector for conantokin-G secretion and an attenuated Salmonella strain transformed therewith. This application claims priority to Korean Patent Application No. 10-2022-0090612, filed on Jul. 21, 2022, in the Korean Intellectual Property Office, the disclosure of which is incorporated herein by reference.

### BACKGROUND ART

Cancer is a disease that ultimately threatens life as cells continue to proliferate, invade surrounding tissues, and destroy normal cells. As the body's regulatory ability declines with age, it becomes more vulnerable to cancer, and in an aging society, cancer remains the leading cause of death, more than double the mortality rate of heart disease, which ranks second.

The most effective response system for treating disease is to strengthen the immunity, but cancer cells are not foreign invaders, so they evade the body's immune response without triggering it. Certain viruses or bacteria tend to infect cancer cells more than normal cells, and infected cancer cells may become targets for immune cell attacks. Accordingly, anticancer treatment methods have been proposed that intentionally infect certain viruses or bacteria to stimulate the human immune response against cancer cells. Infectious bacteria such as Shigella, Vibrioholera, and pathogenic E. coli only invade intestinal cells but do not reach the liver and spleen, which are important organs where immune responses occur. In contrast, Salmonella bacteria may invade the spleen and liver through lymph nodes and stimulate a systemic immune response. Specifically, according to Leschner et al. (J. Mol. Med. 2010, 88, 763-773), CT26 tumor-bearing mice were intravenously with fluorescent Salmonella, and the course of infection over time was tracked. As a result, it was shown that immediately after infection, the whole body of the mouse was infected through the mouse's blood, 20 minutes after infection, Salmonella bacteria accumulated in the spleen and liver, and 24 hours later, Salmonella was observed to have concentrated accumulation exclusively in the tumor tissue.

However, as Salmonella is a representative bacteria that causes food poisoning and can cause sepsis through infection, which is life-threatening, it is too pathogenic to be used directly for cancer treatment. A research team at Yale University in the United States announced that by genetically modifying Salmonella, it is possible to attenuate the bacteria by removing only the toxicity while maintaining the tumor-attacking properties, and that tumors can be suppressed by inducing immune stimulation through injection of the attenuated Salmonella. However, attenuated Salmonella have low survivability and reduced immune-inducing ability, and there is a risk that mutations can cause the attenuated strain to transform into wild-type Salmonella, potentially leading to sepsis. In addition, due to a lack of research and development funding, most anticancer treatments using attenuated Salmonella have been suspended or put on hold in the first phase of clinical trials.

The potential for sepsis associated with cancer treatment using bacteria such as Salmonella is a critical issue that must be overcome, and in addition, the stimulation of the immune response must also be actively achieved. Accordingly, research is mainly focused on developing strains that can additionally express or suppress substances beneficial to anticancer treatment, along with attenuation and immune activation, and using these strains in anticancer treatment. KR Patent No. 10-0852687 discloses a Salmonella strain expressing tumor necrosis factor-alpha by transducing a tumor necrosis factor-alpha protein vector into the attenuated Salmonella strain, and a composition for anticancer treatment containing the same, and KR Publication No. 10-2021-0123556 discloses an anticancer Salmonella that induces an immune response through filament growth regulation.

“Conantokin-G” is a peptide composed of 17 amino acids and is a toxin secreted by sea snails belonging to the Cone Snail genus. It is known as an antagonist of N-methyl-D-aspartate receptor (NMDAR) and is known as a sleeper peptide. It has been reported to have potential as a neuroprotective agent in ischemia, excitotoxicity, and pain, and as a research tool for drug addiction and Alzheimer's disease. It is also known to reduce the intensity of excitotoxic intracellular calcium ions and block nerve damage. In particular, NMDAR is associated with cancer growth, and is emerging as a target for tumor treatment.

Accordingly, the present inventors prepared a genetic construct linking the flgM gene and a gene encoding the conantokin-G protein, and introduced the genetic construct into an attenuated Salmonella strain. This enabled the secretion of conantokin-G together with flgM, thereby confirming that in addition to the inherent cancer cell targeting and anticancer activity of Salmonella, the anticancer effect can be further maximized by the action of a large amount of conantokin-G possessing anticancer activity, and thus the present disclosure was completed based on this.

The information set forth in this Background Art section is intended solely to enhance understanding of the background of the present disclosure and may not include information that constitutes prior art, already known to a person skilled in the art to which the present disclosure pertains.

### DISCLOSURE OF INVENTION

### Technical Problem

An object of the present disclosure is to provide a recombinant expression vector for effectively secreting the conantokin-G protein exhibiting excellent anticancer effects, and an attenuated Salmonella strain transformed with the expression vector.

Another object of the present invention is to provide an anticancer pharmaceutical composition including the attenuated Salmonella strain as an active ingredient.

### Solution to Problem

An aspect is to provide an attenuated Salmonella strain transformed with a recombinant expression vector including a flgM gene and a conantokin-G gene.

As used herein, “flgM” refers to a protein (or a gene encoding such a protein) that inhibits the σ factor, which is responsible for recruiting RNA polymerase required for late flagellar transcription in a Type III secretion system of bacteria. As used herein, the “Type III secretion system” is a pathogenic substance delivery method developed in Gram-negative bacteria, and it refers to a multi-protein complex, a syringe-shaped structure that injects pathogenic active proteins directly into the cytoplasm through the host cell membrane, (Mota L J et al, Ann Med. (2005); 37 (4): 234-249). In the Type III secretion system, the flgM protein remains in the cytoplasm during the formation of the hook-basal body, the basic structure capable of forming the flagellum on the cell membrane, and acts as an anti-σ²⁸ factor that inhibits the transcription of class III genes, such as the flagellin subunit FliC or the stator protein MotAB. Subsequently, after completion of the hook-basal body structure, a change in substrate specificity occurs simultaneously within the flagellar secretion system, causing flgM to be secreted out of the cell through the central channel of the hook, and subsequently initiating σ²⁸-dependent transcription within the cell. The secretion mechanism of flgM through the flagellar formation process in the Type III secretion system is shown in FIG. 2.

Specifically, the present disclosure produced a genetic construct including the flgM gene and the conantokine-G gene, which is a protein to be secreted, thereby enabling secretion of conantokine-G outside the strain together with the secretion of flgM via the strain's Type III secretion system.

The term “conantokin-G” as used herein is a peptide composed of 17 amino acids, a toxin secreted by Conus geographus, a genus of Cone snail. The sequence of conantokin-G can be obtained from the publicly available database, NCBI GeneBank.

As used herein, the term “gene” is to be considered in the broadest sense, and may encode a structural protein or a regulatory protein. The regulatory protein may include a transcription factor, a heat shock protein, or a protein involved in DNA/RNA replication, transcription and/or translation.

As used herein, the term “transformation” refers to introducing a vector containing a polynucleotide encoding a target protein into a host cell, enabling the expression of the protein encoded by the polynucleotide within the host cell. For example, the polynucleotide may be introduced into a host cell in the form of an expression cassette, or may be introduced into a host cell in its own form and operably linked to a sequence necessary for expression in the host cell, but is not limited thereto. According to an embodiment, a recombinant expression vector containing the flgM gene and the conantokin-G gene, which is a protein to be secreted, is constructed, and by transforming a Salmonella strain with the vector, enabling not only the expression of conantokin-G but also the regulation of the secretion of conantokin-G outside the cell.

The term “attenuation” as used herein refers to artificially weakening the virulence of a living pathogen by mutating genes involved in essential metabolism of the pathogen so that it cannot cause disease in the body but only stimulates the immune system, thereby inducing immunity. Genes causing attenuation of Salmonella are well known in the art, and for example, the attenuated Salmonella strain may be one in which the function of one or more genes selected from the group consisting of aroA, aroC, aroD, aroE, asd, Rpur, htrA, ompR, ompF, ompC, galE, cya, crp, cyp, phoP, phoQ, rfaY, dksA, hupA, sipC, clpB, clpP, clpX, pab, nadA, pncB, pmi, rpsL, hemA, rfc, poxA, galU, cdt, pur, ssa, guaA, guaB, fliD, flgK, flgL, relA, and spoT, has been inactivated. To prevent reversion to the wild type and to further weaken virulence during in vivo application, the function of two or more of the genes may be inactivated. According to an embodiment, the attenuated Salmonella strain may be a Salmonella strain in which the aroA, aroD and asd genes are deleted.

The attenuated Salmonella strain is targeted to tumor sites, where it induces an immune response, thereby exhibiting anticancer activity. Meanwhile, when transformed with the recombinant expression vector of the present disclosure, the Salmonella strain is targeted to the tumor sites, and display enhanced secretion capacity of conantokin-G, for example, an flgM-conantokin-G fusion protein, thereby exhibiting more potent anticancer activity.

In an embodiment, the flgM gene and the conantokin-G gene may be linked by a linker. As used herein, the term “linker” refers to a polypeptide chain used to connect a heterologous domain to a protein of interest, and in an embodiment, the linker may be, for example, any one selected from the group consisting of, but is not limited to, (GₗSₘ)ₙ (wherein l, m and n are each ≥2), (GₗSₘ)ₙ-Hₚ-(GₗSₘ)ₙ (wherein l, m and n are each ≥1 and H≥6), (G₄S)ₐ(EAAAK)_{b}(G₄S)ₐ (wherein a and b are integers from 1 to 4), (G₄S)ₚ(EAAAK)_{q} (wherein p and q are integers from 1 to 4), (EAAAK)ₓ(G₄S)_{y} (wherein x and y are integers from 1 to 4), A(EAAAK)₄ALEA(EAAAK)₄A, (GSSGGS)ᵢ (wherein i is an integer from 1 to 4), KESGSVSSEQLAQFRSLD, EGKSSGSGSESKST, GSAGSAAGSGEF, (EAAAK)ₖ (k is an integer from 1 to 5), CRRRRRREAEAC, GGGGGGGG, GGGGGG, AEAAAKEAAAAKA, PAPAP, VSQTSKLTRAETVFPDV, PLGLWA, TRHRQPRGWE, AGNRVRRSVG, RRRRRRRR, and GSSGGSGSSGGSGGGDEADGSRGSQKAGVDE.

The linker may be, for example, a peptide linker of 10 to 16-mer, and the length of the peptide may be a peptide linker of 10 to 15-mer, 10 to 14-mer, 10 to 13-mer, 10 to 12-mer, 10 to 11-mer, 12 to 16-mer, 12 to 15-mer, 12 to 14-mer, 12 to 13-mer, 13 to 16-mer, 13 to 15-mer, or 13 to 14-mer. The length of the linker may affect the secretion level of the flgM-conantokin-G fusion protein of the Salmonella strain according to an embodiment.

In an embodiment, the attenuated Salmonella strain may have enhanced conantokin-G secretion capacity. In an embodiment, the attenuated Salmonella strain may be transformed with a vector containing an arabinose-inducible promoter, enabling the regulation of conantokin-G expression in the presence of arabinose, and using the flgM of the Type III secretion system allow the efficient secretion of conantokin-G protein outside the strain.

In an embodiment, the attenuated Salmonella strain may further include the flhDC gene. The flhDC gene is a main regulator of the flagellin operon genes and can regulate the expression of the flgM gene. Therefore, when the flhDC gene is included in the genetic construct, it may contribute to increasing the expression or secretion levels of flgM-conantokin-G.

In an embodiment, the attenuated Salmonella strain may be a Salmonella strain deposited under accession number KCTC15483BP. According to an embodiment, the attenuated Salmonella strain was transformed with a recombinant expression vector containing a flgM gene-linker-conantokin-G-flhDC gene construct, wherein the gene construct was capable of enhancing the secretion capacity of flgM-conantokin-G of the attenuated Salmonella strain while maintaining the inherent anticancer activity of conantokin-G. Therefore, the transformed attenuated Salmonella strain according to an embodiment may be utilized as an active ingredient for cancer treatment.

Another aspect provides an anticancer pharmaceutical composition or a pharmaceutical composition for preventing or treating cancer including the attenuated Salmonella strain as an active ingredient.

The terms or elements mentioned in the description of the attenuated Salmonella strain that are identical to those already mentioned are as described above.

“Cancer,” a disease targeted for prevention or treatment by the composition of the present disclosure, is a general term for a disease caused by cells that have aggressive characteristics in which cells divide and grow while disregarding normal growth limits, invasive characteristics in which cells infiltrate surrounding tissues, and metastatic characteristics in which cells spread to other parts of the body. The cancer may be, for example, a solid tumor, and preferably, a tumor including cancer tissue or cancer cells in which the cancer-targeting ability of the Salmonella strain can be exerted, and thus, it may be broadly applied without limitation. For example, the types of cancer may be any one selected from the group consisting of pancreatic cancer, colorectal cancer, colon cancer, stomach cancer, liver cancer, colorectal cancer, brain cancer, breast cancer, thyroid cancer, bladder cancer, esophageal cancer, uterine cancer, and lung cancer, but is not limited thereto.

The composition of the present disclosure can be manufactured by methods known in the pharmaceutical field for use as an anticancer agent, and can be used on its own or mixed with a pharmaceutically acceptable carrier, forming agent, diluent, etc.

As used herein, “pharmaceutically acceptable carrier” may be determined partly by the specific composition being administered, as well as partly by the specific method used for administering the composition. Accordingly, suitable formulations of the compositions are highly variable, and the compositions may be formulated, for example, for parenteral (i.e., intramuscular, intradermal, or subcutaneous) administration or nasopharyngeal (i.e., intranasal) administration. Generally, formulations for parenteral administration include sterile aqueous or non-aqueous solutions, suspensions and emulsions. Examples of non-aqueous solvents include propylene glycol, polyethylene glycol, vegetable oils such as olive oil, and injectable organic esters such as ethyl oleate. Aqueous carriers may include water, alcohol/aqueous solutions, emulsions or suspensions, including saline and buffered media. Parenteral vehicles may include sodium chloride solution, Ringer's dextrose, dextrose and sodium chloride, lactated Ringer's, or fixed oils. Preservatives and other additives may also be present, such as, for example, antimicrobial agents, antioxidants, chelating agents, and inert gases.

The dosage of the active ingredient according to the present disclosure can be appropriately selected depending on the degree of absorption of the active ingredient in the body, the form of the preparation, the age, gender and condition of the patient, the degree of symptoms, etc., and the administration can be once a day or divided into several times. The typical dosage is from 0.001 mg/kg·day to 10 g/kg·day.

Another aspect provides a method of preventing or treating cancer, including administering to a subject the pharmaceutical composition in an amount effective to prevent or treat the cancer.

Any terms or elements mentioned in the description of the pharmaceutical composition that are identical to those already mentioned are as described above.

The subject may be a mammal. The mammal may be humans, mice, rats, dogs, cats, horses, cows, goats, or pigs.

As used herein, the terms “treatment” or “treating” or “alleviating” or “improving” are used interchangeably. These terms refer to methods of obtaining beneficial or desired results, including but not limited to therapeutic benefits and/or preventive benefits. Therapeutic benefits refer to any therapeutically significant improvement or effect on one or more diseases, conditions or symptoms under treatment. For preventive benefits, the composition may be administered to a subject at risk of developing a particular disease, condition or symptom, or to a subject reporting one or more physiological symptoms of a disease, even if the disease, condition or symptom has not yet manifested.

The pharmaceutical composition may be administered in single or multiple doses in a pharmaceutically effective amount. The composition may be formulated and administered into forms of liquids, powders, aerosols, injections, intravenous solutions (Ringer's solution), capsules, pills, tablets, suppositories, or patches. The route of administration of the pharmaceutical composition for preventing or treating cancer by administering the anticancer agent may be any general route as long as it can reach the target tissue.

The pharmaceutical composition may be administered via a route such as intraperitoneal administration, intravenous administration, intramuscular administration, subcutaneous administration, intradermal administration, transdermal patch administration, oral administration, intranasal administration, intrapulmonary administration, or rectal administration, depending on the intended purpose, but is not particularly limited thereto.

Another aspect provides: a genetic construct including the flgM gene and the conantokin-G gene; or a recombinant expression vector including the flgM gene and the conantokin-G gene.

The terms or elements already mentioned in the description of flgM and conantokine-G are as described above.

As used herein, the term “genetic construct” refers to a collection, functional unit, or construct containing genetic information of a target protein, and may be interpreted in the broadest sense. The genetic construct, for the purposes of the present disclosure, is intended to enhance the secretion of the flgM-conantokin-G fusion protein from attenuated Salmonella strain, and for example, it may refer to a DNA insert in which the flgM gene, a 10 to 16-mer peptide linker, and the conantokin-G gene are operatively linked.

As used herein, the term “vector” refers to a DNA construct containing a nucleotide sequence encoding a target polynucleotide operably linked to suitable regulatory sequences so as to enable expression of the target polynucleotide in a suitable host. The regulatory sequence may include a promoter capable of initiating transcription, an optional operator sequence for regulating such transcription, a sequence encoding a suitable mRNA ribosome binding site, and a sequence regulating the termination of transcription and translation. After being transformed into a suitable host, the vector can replicate and function independently of the host genome, or it may be integrated into the genome itself. The vector may be any one capable of replication in a host cell. For example, the vector may be a natural or recombinant plasmid, cosmid, virus or bacteriophage. The vector may include a selectable marker for selecting a host cell containing the vector. Selectable markers are used to identify cells transformed with the vector, and markers that confer a selectable phenotype, such as drug resistance, nutrient requirement, resistance to cytotoxic agents, or expression of surface proteins, may be used. For example, the marker may be one or more selectable markers selected from the group consisting of ampicillin, neomycin, puromycin, hygromycin and zeocin. The vector may include genes involved in replication and/or copy number control, such as a replication origin, a promoter, and may include, but is not limited to, a restriction enzyme site.

In an embodiment, the recombinant expression vector may be operably linked to an inducible promoter. As used herein, an “inducible promoter” is a promoter capable of switching the operation of a linked gene by an inducer, and examples include an ara promoter, a tac promoter, a lac promoter, a lacUV5 promoter, a lpp promoter, a pLA promoter, a pRA promoter, a rac5 promoter, an amp promoter, a recA promoter, a SP6 promoter, a trp promoter, a T7 promoter, a pBAD promoter, a Tet promoter, a trc promoter, a pepT promoter, a sulA promoter, a pol 11(dinA) promoter, a ruv promoter, a uvrA promoter, a uvrB promoter, a uvrD promoter, a umuDC promoter, a lexA promoter, a cea promoter, a caa promoter, a recN promoter, a pagC promoter, a hip promoter, ansB promoter or pflE promoter.

As used herein, “operably linked” may refer to nucleotide sequences are connected on a single nucleic acid fragment such that one function is affected by the other. In an embodiment, a recombinant expression vector of flgM-(linker)-conantokin-G-pBAD18, in which the gene is operably linked to the ara promoter, was established using the pBAD18 asd plasmid as a vector to express the flgM-conantokin-G protein. The asd gene may be composed of a nucleotide sequence of SEQ ID NO: 10.

In an embodiment, a Shine-dalgarno sequence may be inserted upstream of the genetic construct of the present disclosure to improve the expression efficiency of the conantokin-G. In addition, appropriate restriction enzymes may be placed at the upstream and downstream regions of the genetic construct of the present disclosure so that the genetic construct of the present disclosure may be introduced into a vector.

In an embodiment, the flgM gene may be composed of a nucleotide sequence of SEQ ID NO: 1, and/or the conantokin-G gene may be composed of a nucleotide sequence of SEQ ID NO: 2. Furthermore, the recombinant expression vector may additionally include the flhDC gene, and the flhDC gene may be composed of a nucleotide sequence of SEQ ID NO: 3.

As used herein, “variants” refer to entities that exhibit significant structural identity with a reference entity (e.g., a wild type sequence) but are structurally different from the reference entity in one or more aspects. Whether a particular entity is appropriately considered a “variant” of a reference entity is based on the degree of structural or functional identity to the reference entity. Any biological or chemical reference entity has certain unique structural elements, and a variant, by definition, refers to a distinct chemical entity that shares one or more of those unique structural elements. For example, a polynucleotide may have unique sequence elements including a plurality of nucleotide residues having specified positions relative to one another in linear or three-dimensional space. For example, a polynucleotide may differ from a reference polynucleotide by one or more differences in the nucleotide sequence and/or by one or more differences in chemical moieties (e.g., carbohydrates, lipids, etc.) covalently linked to the polynucleotide backbone. In some embodiments, the variant exhibits an overall sequence identity to a reference polynucleotide of at least 60%, 65%, 70%, 75%, 80%, 85%, 86%, 87%, 88%, 89%, 90%, 91%, 92%, 93%, 94%, 95%, 96%, 97%, 98% or 99%.

In an embodiment, the expression of the flgM gene; the conantokin-G gene; and the flhDC gene may be regulated by the same inducible promoter. As the expression of the flgM gene; the conantokin-G gene; and the flhDC gene are regulated by the same inducible promoter, the secretion capacity of the flgM and conantokin-G proteins outside the strain may be enhanced, thereby enhancing the anticancer effect of a composition including the strain.

### Advantageous Effects of Invention

The attenuated Salmonella strain according to the present disclosure is transformed with a recombinant expression vector including a flgM gene-linker-conantokin-G gene construct and the flhDC gene, thereby inducing high levels of conantokin-G expression and secretion. Therefore, the recombinant expression vector according to an aspect, the attenuated Salmonella strain transformed with the vector, and the anticancer pharmaceutical composition including the vector can be utilized to treat cancer.

### BRIEF DESCRIPTION OF DRAWINGS

FIG. 1 is a schematic diagram showing the flgM-conantokin-G protein secretion process using the Type Ill secretion system.

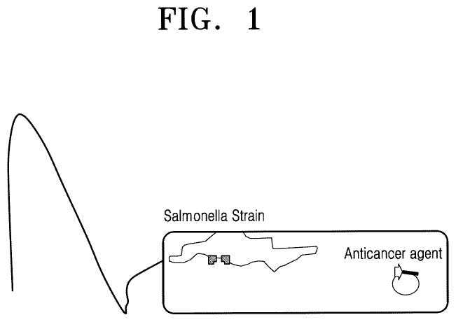

FIG. 2 is a schematic diagram showing the flgM secretion mechanism using the flagellar formation process in the Type III secretion system.

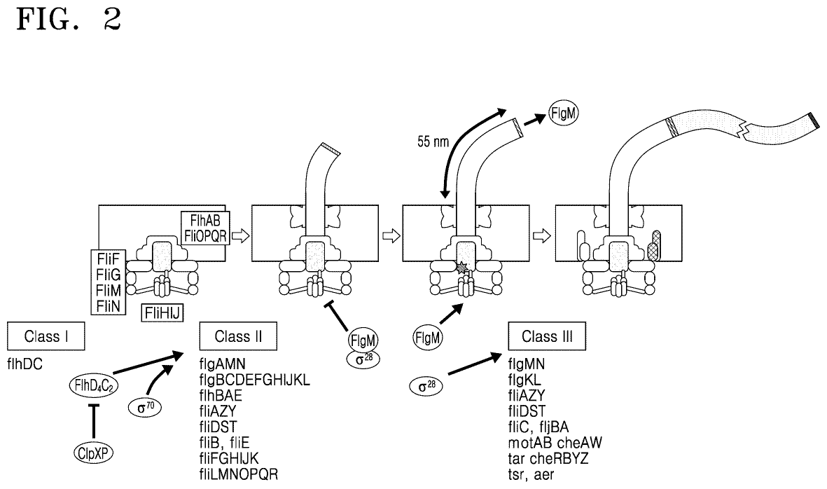

FIG. 3 is a diagram showing a cleavage map of the flgM-linker-conantokin-G-flhDC pBAD18 plasmid according to an embodiment.

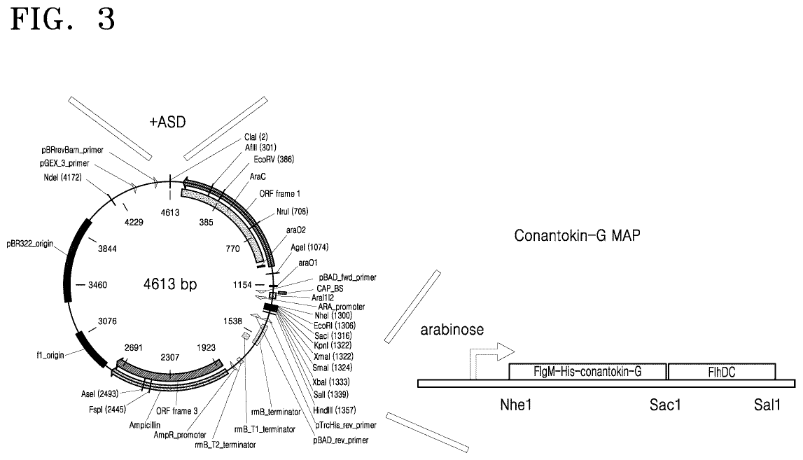

FIG. 4 shows the results of demonstrating the protein secretion level of an attenuated Salmonella strain transformed with the flgM-linker-conantokin-G-flhDC pBAD18 plasmid according to an embodiment.

FIG. 5 shows the results of evaluating the activity of a flgM-conantokin-G fusion protein secreted from an attenuated Salmonella strain according to an embodiment, demonstrating the antitumor effect against colon cancer cell lines.

FIG. 6 shows the results of evaluating the activity of a flgM-conantokin-G fusion protein secreted from an attenuated Salmonella strain according to an embodiment, evaluating the level of cancer cell death through CCK assay and LDH assay.

FIG. 7 shows the results of quantitatively demonstrating the change in tumor volume over time after intravenous administration of the flgM-conantokin-G/aroA aroD asd-strain according to an embodiment to a colon cancer mouse model.

FIG. 8 shows the results of demonstrating the change in tumor volume over time for each individual after intravenous administration of the flgM-conantokin-G/aroA aroD asd-strain according to an embodiment to a colon cancer mouse model.

FIG. 9 shows the results of visually demonstrating changes in tumor tissue over time after intravenous administration of the flgM-conantokin-G/aroA aroD asd-strain according to an embodiment to a colon cancer mouse model.

### MODE FOR THE INVENTION

The following examples will be described in more detail. However, these examples are provided for illustrative purposes only and are not intended to limit the scope of the present disclosure.

### Example 1: Construction of a Plasmid for Conantokin-G Secretion

1-1. Construction of Genetic Construct Linked with flgM Gene and Conantokin-G Gene

The flgM DNA sequence of wild-type Salmonella (flgM anti-sigma-28 factor FlgM [Salmonella enterica subsp. enterica serovar Typhimurium str. LT2] Gene ID: 1252690, Locus tag STM1172 NC_003197.2 (1257036 . . . 1257341, complement)) and a DNA sequence obtained through codon optimization for conantokin-G were linked to produce a genetic construct according to an embodiment. Specifically, the flgM DNA sequence and the conantokin-G DNA sequence were connected via a linker, and to enhance the expression of the flgM gene, a 14-base Shine-Dalgarno sequence (AGGAGGTTTGACCT) was inserted upstream of the flgM gene. Additionally, for cloning purposes, a Nhe I site was inserted at the 5′ end, and a Sac I site was inserted after the stop codon of conantokin-G. Meanwhile, the genetic information of the main structures in this example is as shown in Table 1 below.

TABLE 1

| | | Sequence |
|---|---|---|
| Name | Sequence Information | Number |
| flgM | ATGAGCATTGACCGTACCTCACC | 1 |
| | TTTGAAACCCGTTAGCACTGTCC | |
| | AGACGCGCGAAACCAGCGACACG | |
| | CCGGTACAAAAAACGCGTCAGGA | |
| | AAAAACGTCCGCCGCGACGAGCG | |
| | CCAGCGTAACGTTAAGCGACGCG | |
| | CAAGCGAAGCTCATGCAGCCAGG | |
| | CGTCAGCGACATTAATATGGAAC | |
| | GCGTCGAAGCATTAAAAACGGCT | |
| | ATCCGTAACGGTGAGTTAAAAAT | |
| | GGATACGGGAAAAATAGCAGACT | |
| | CGCTCATTCGCGAGGCGCAGAGC | |
| | TACTTACAGAGTAAAAAA | |
| conantokin- | GGCGAAGAGGAATTACAGGAAAA | 2 |
| G | CCAGGAATTGATCCGTGAAAAAT | |
| | CCAAT | |
| linker 1 | CATCACCATCACCATCAC | 4 |
| Linker 2 | GGCGGCAGCAGCCATCACCATCA | 5 |
| | CCATCACAGCAGCGGCGGC | |

Using the genomic DNA of wild-type Salmonella LT2 as a template, and the primers flhD-sac1-F and flhC-Sal1-r in Table 2, a PCR product containing the flhDC gene was obtained by PCR. The specific PCR conditions are as follows: 5 μl of 10× buffer, 10 μl of 5×Q solution, 7.5 μl of 2 mM dNTP, 2.5 μl of 20 μM flhD-sac1-F primer, 2.5 μl of 20 μM flhC-Sal1-r primer, 1 μl of Taq polymerase, 2 μl of 10 ng/μl genomic DNA, and 19.5 μl of water were mixed and PCR was performed using the Qiagen system under the following conditions: 95 °C for 5 minutes, followed by 30 cycles of 94 °C for 30 seconds, 49 °C for 30 seconds, and 72 °C for 1 minute, and then 72 °C for 10 minutes. 1 μg of PCR fragment purified using a PCR purification kit was reacted with 10 U each of sac I and Sal I at 37 °C for 2 hours, and the reaction product was purified using a PCR purification kit to produce flhDC insert DNA of SEQ ID NO: 3.

TABLE 2

| | | Sequence |
|---|---|---|
| Name | Sequence Information | Number |
| flhD- | gatcgatcgagctcaggaggtttg | 8 |
| SAC1-f | atcctatgggaacaatgcatacat | |
| | ccgagttgct | |
| flhC- | gatcgatcgtcgacttaaacagcc | 9 |
| Sal1-r | tgttcgatctgttcatccagcagt | |
| | t | |

After reacting for 2 hours at 37 °C, the reaction products were purified using a PCR purification kit to obtain each vector DNA. The asd gene inserted in the plasmid may be composed of a nucleotide sequence of SEQ ID NO: 10. Afterwards, each vector DNA was ligated with the flgM-conantokin-G insert DNA of Example 1-1 at 25 °C for 30 minutes, and transformed into DH5a competent cells. Thereafter, the transformed cells were plated onto LB amp solid medium and cultured at 37 °C to select colonies with antibiotic resistance. Six candidates from the selected colonies were selected and reacted with 10 U of sac I and Sal I at 37 °C for 1 hour, and then the generation of 400 bp bands was confirmed through electrophoresis. Thereafter, nucleotide sequence analysis of the candidate group was performed on the plasmids constructed using pBAD18 to confirm the insertion of the flgM, linker1, linker2, and conantokin-G genes, and the flgM-linker1-conantokin-G-pBAD18 plasmids and flgM-linker2-conantokin-G-pBAD18 plasmids were established according to an embodiment.

Afterwards, 1 μg of the constructed flgM-linker1-Conantokin-G-pBAD18 plasmid or flgM-linker2-Conantokin-G-pBAD18 plasmid was reacted with 10 U each of sac I and Sal I at 37 °C for 2 hours, and the reaction products were purified using a PCR purification kit to complete the vector DNA. Each vector DNA was then ligated with the flhDC insert DNA of Example 1-2 at 25 °C for 30 minutes, and transformed into DH5α (DH5a) competent cells. The transformed cells were plated onto LB amp solid medium and cultured at 37 °C to select colonies with antibiotic resistance. Six candidates from the selected colonies were selected and reacted with 10 U of sac I and Sal I at 37 °C for 1 hour, and then the generation of bands was confirmed through electrophoresis. A nucleotide sequence analysis of the candidate group was performed to confirm the insertion of the flhDC gene, and the flgM-linker1-conantokin-G-pBAD18 plasmids and flgM-linker2-conantokin-G-pBAD18 plasmids were established.

### Example 2: Construction of Attenuated Salmonella Strain Secreting flgM-Conantokin-G

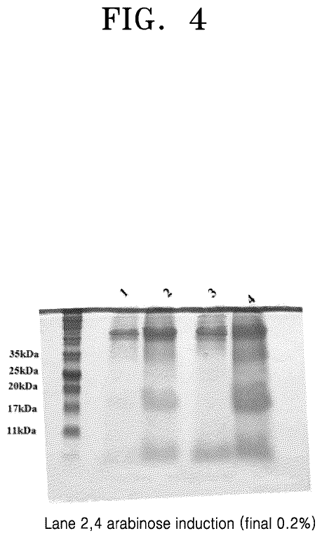

An attenuated Salmonella strain was transformed using the plasmid prepared in Example 1. The attenuated Salmonella strain used was aro A aro D asd-strain, and the attenuated Salmonella strain was controlled to regulate the expression of conantokin-G by arabinose and to secrete it outside the cell. A single colony of each of the transformed strains was inoculated into LB amp liquid medium and cultured with shaking at 37 °C for 12 hours to 16 hours. The culture was then diluted in fresh LB amp liquid medium to an OD600 of 0.05 and cultured with shaking at 37 °C for 2 hours. When the OD600 reached 0.4 to 0.6 by shaking culture, arabinose was added at a concentration of 0.2 w % and the culture was shaken at 37 °C for additional 5 hours. Afterwards, the culture solution was centrifuged at 3000 rpm to separate the supernatant, and filtered through a 0.2 μm filter to completely remove the bacteria. 50 μl of the supernatant from which the bacteria had been removed was mixed with SDS sample buffer and denatured for 5 minutes in a 100 °C heating block. Then, centrifugation was performed for 5 minutes at 13,000 rpm and 4 °C, and electrophoresis was performed on a 10% polyacrylamide gel. After electrophoresis at 100 V for 1 hour and 30 minutes, Western blotting was performed by staining with Coomassie Brilliant Blue G 250 and destaining with distilled water. Through this, the amount of flgM-conantokin-G in the culture medium was confirmed and shown (FIG. 4). In FIG. 4, lanes 1 and 2 are the group transformed with the flgM-linker1-Conantokin-G-flhDC pBAD18 plasmid (flgM-linker1-Conantokin-G-flhDC pBAD18/aroA aro D asd-), lanes 3 and 4 are the group transformed with the flgM-linker2-Conantokin-G-flhDC pBAD18 plasmid (flgM-linker2-Conantokin-G-flhDC pBAD18/aroA aro D asd-), and lanes 2 and 4 are the group in which expression was induced by treatment with arabinose, unlike lanes 1 and 3.

As a result, as shown in FIG. 4, lanes 2 and 4 treated with arabinose secreted higher amount of flgM-conantokin-G compared to lanes 1 and 3 not treated with arabinose at the same concentration, and among them, the secretion amount in lane 4 to which a specific linker was applied was very significant compared to lane 2. From these results, it was found that the strain transformed with the flgM-linker-Conantokin-G-flhDC-pBAD18 plasmid according to an embodiment exhibited excellent conantokin-G secretion capacity, and in particular, the secretion capacity of the flgM-linker2-Conantokin-G-flhDC pBAD18/aroA aro D asd-strain to which a 14mer linker was applied was very excellent. Accordingly, the inventors deposited a strain flgM-linker2-Conantokin-G-flhDC pBAD18/aroA aro D asd-according to an embodiment to the Biological Resource Center of the Korea Research Institute of Bioscience and Biotechnology on Jun. 26, 2023, and were assigned the accession number KCTC15483BP (S-flgM-Conantokin-pBAD18-ASD+/BRD509 asd−).

### Example 3: Testing of In Vitro Antitumor Effects of Conantokin-G

3-1. Evaluation of Activity of Secreted flgM-Conantokin-G

A single colony of the transformed flgM-linker2-Conantokin-G-flhDC pBAD18/aroA aro D asd-strain obtained in Example 2 was inoculated into LB amp liquid medium and cultured with shaking at 37 °C for 12 hours to 16 hours. The culture was then diluted in fresh LB amp liquid medium to an OD600 of 0.05 and cultured with shaking at 37 °C for 2 hours. When the OD600 reached 0.4 to 0.6 by shaking culture, arabinose was added at a concentration of 0.2 w % and the culture was shaken at 37 °C for an additional 5 hours. For comparison, a control group not treated with arabinose was cultured under the same shaking conditions. The culture solution was centrifuged at 3000 rpm to separate the supernatant, and the supernatant was filtered through a 0.2 μm filter to completely remove the bacteria. Through this, a flgM-conantokin-G fusion protein was obtained.

Separately, murine CT26 mouse colon cancer cell line, and human colon cancer cell lines HT29 and SW480 were purchased, and each cell was grown in Dulbecco's modified Eagle's medium (DMEM) containing 10% fetal bovine serum (FBS) and 1% penicillin-streptomycin, and the cells were seeded in 6-wells when the number of cells was 2×10⁵. After 24 hours, 200 μl of the fusion protein obtained from the supernatant through the above process was added, and the cells were observed after 48 hours of incubation.

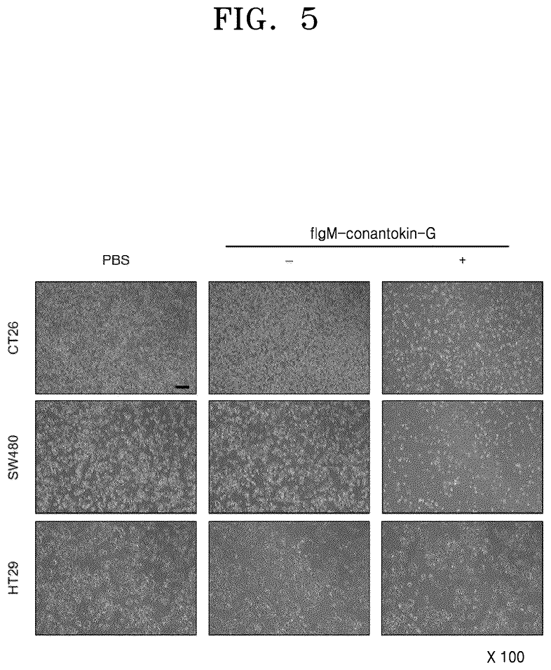

As a result, as shown in FIG. 5, the control group (−) that was not treated with arabinose showed a decrease in cell level by about 5% compared to the control group treated with PBS, whereas the experimental group (+) that was treated with arabinose to induce expression or secretion of flgM-conantokin-G showed a decrease in cell level by more than 50%.

### 3-2. Evaluation of Cancer Cell Apoptosis Induction Effect

To confirm the effect on inducing cancer cell apoptosis, CCK assay and LDH assay were performed using the culture medium and cells of Example 3-2. For the CCK assay, 100 μl of the supernatant from the cultured cell lines was transferred to a 96-well plate, followed by the addition of 100 μl of CCK solution, and measurement was performed using a 450 nm microreader. This experiment was conducted in triplicate to increase reliability. In addition, for the LDH assay, the supernatant was centrifuged at 1000 rpm for 3 minutes to remove cell debris, and 10 μl of the supernatant was mixed with 100 μl of LDH solution. The mixture was incubated at room temperature for 30 minutes, followed by measurement using a 450 nm microreader. As with the CCK assay, the experiment was conducted in triplicate.

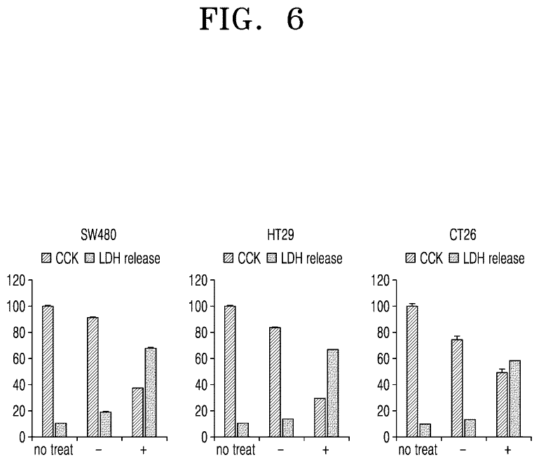

As a result, as shown in FIG. 6, the experimental group (+) that induced the expression or secretion of flgM-conantokin-G had the lowest level of detected cell activity and the highest level of cells lysis, compared to the untreated control or the group not treated with arabinose (−). These results experimentally demonstrate that the attenuated Salmonella strain transformed according to an embodiment retains the inherent cell-killing ability of conantokin-G, while its secretion capability is enhanced through the unique structure linked to the Type III secretion system with flgM.

### Example 4: Evaluation of Antitumor Effects Using Mouse Colon Cancer Model

In this example, female BALB/c mice aged 6 weeks and weighing around 20 g were purchased and used as experimental animals. All animal care, experiments, and euthanasia were performed according to approved protocols. Murine CT26 colon carcinoma cells were purchased from the American Type Culture Collection (ATCC) and cultured in DMEM medium containing 10% FBS and 1% penicillin-streptomycin. CT26 tumor cells cultured in vitro were harvested and suspended in 20 μl of PBS, and 1×10⁶ cells were injected subcutaneously into the right back of BALB/c mice to perform xenografting of the tumor cells. About 2 weeks after the transplantation of tumor cells, when the tumor size reached 100 mm³, the flgM-linker2-conantokin-G-flhDC pBAD18/aroA aro D asd-strain produced in Example 2 was diluted with PBS and injected through the tail vein at a dose of about 5×10⁷ CFU/mice (0 day). Subsequently, to induce the expression or secretion of flgM-conantokin-G in the mice, 100 μl/mice of 40% L-arabinose was intraperitoneally injected daily, and the weight and tumor size of each mouse were measured and recorded daily at a consistent time. The tumor volume was calculated using the formula (tumor length×tumor width×tumor height)/2, and mice were euthanized when the tumor volume exceeded 1,500 mm³ in accordance with the guidelines of the Animal Experiment Ethics Committee. For the control groups, the following were used: a group injected with only PBS buffer (PBS), a comparison group injected with the attenuated aroA aroD asd-Salmonella strain (Salmonella), a group injected with the aroA aroD asd-strain transformed with flgM asd (Salmonella+flgM), and a group treated with the strain developed in an embodiment but not treated with arabinose (Co−).

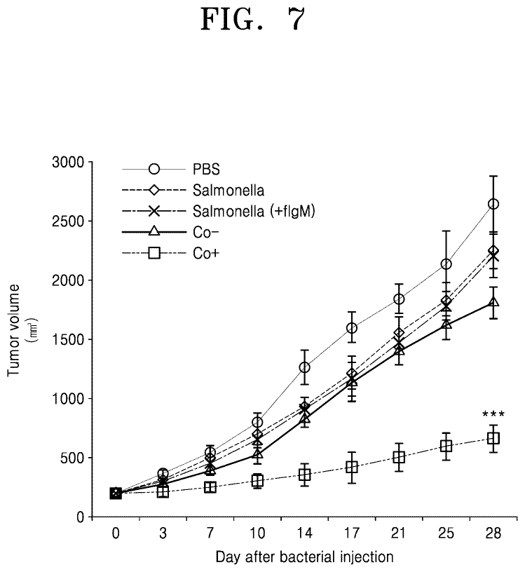

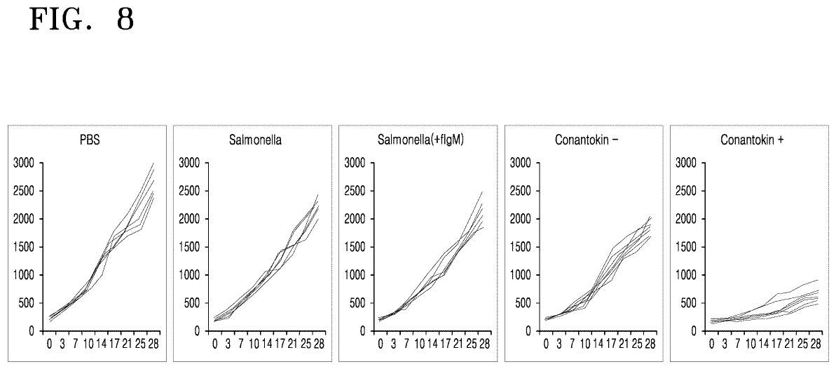

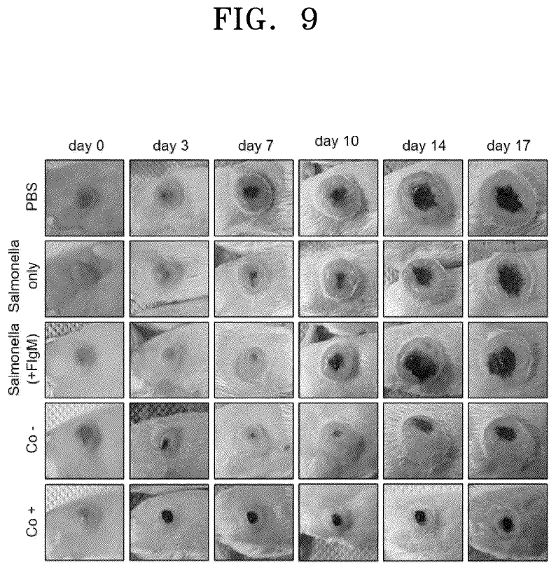

As shown in FIGS. 7 to 9, the group in which the expression or secretion of flgM-conantokin-G was induced by injection of the transformed attenuated Salmonella strain (Co+) according to an embodiment showed a significant inhibition of tumor growth. In particular, this antitumor effect was very significant compared to the groups injected with Salmonella strains (Salmonella, Salmonella+flgM, Co−). From this, it was found that the flgM-linker2-Conantokin-G-flhDC pBAD18/aroA aro D asd-strain according to an embodiment can be utilized as an effective ingredient for treating cancer.

### Accession Number

INTERNATIONAL DEPOSITARY AUTHORITY: Korean Collection for Type Culture (KCTC)
ACCESSION NUMBER: KCTC15483BP
ACCESSION DATE: 20230626

1. An attenuated Salmonella strain transformed with a recombinant expression vector that comprises: a flgM gene; and a conantokin-G gene.

2. The attenuated Salmonella strain of claim 1, wherein the flgM gene and the conantokin-G gene are linked via a linker.

3. The attenuated Salmonella strain of claim 1, wherein the recombinant expression vector further comprises a flhDC gene.

4. The attenuated Salmonella strain of claim 1, wherein the attenuated Salmonella strain is one in which the function of one or more genes selected from the group consisting of aroA, aroC, aroD, aroE, asd, Rpur, htrA, ompR, ompF, ompC, galE, cya, crp, cyp, phoP, phoQ, rfaY, dksA, hupA, sipC, clpB, clpP, clpX, pab, nadA, pncB, pmi, rpsL, hemA, rfc, poxA, galU, cdt, pur, ssa, guaA, guaB, fliD, flgK, flgL, relA, and spoT, is inactivated.

5. The attenuated Salmonella strain of claim 1, wherein the attenuated Salmonella strain has an enhanced secretion capacity for conantokin-G.

6. The attenuated Salmonella strain of claim 1, wherein the attenuated Salmonella strain is deposited under Accession Number: KCTC15483BP.

7. An anticancer pharmaceutical composition comprising the attenuated Salmonella strain of claim 1 as an active ingredient.

8. A recombinant expression vector comprising: a flgM gene; and a conantokin-G gene.

9. The recombinant expression vector of claim 8, wherein the flgM gene and the conantokin-G gene are linked via a linker.

10. The recombinant expression vector of claim 8, wherein the recombinant expression vector further comprises a flhDC gene.

11. The recombinant expression vector of claim 8, wherein the flgM gene consists of a nucleotide sequence of SEQ ID NO: 1.

12. The recombinant expression vector of claim 8, wherein the conantokin-G gene consists of a nucleotide sequence of SEQ ID NO: 2.

13. The recombinant expression vector of claim 8, wherein the flgM gene, the conantokin-G gene, and the flhDC gene are expressed and regulated by a common inducible promoter.


SEQUENCE LISTING (10 sequences)

SEQ ID NO: 1 - DNA, 294 bp, synthetic construct
  source 1..294: mol_type = other DNA
atgagcattgaccgtacctcacctttgaaacccgttagcactgtccagacgcgcgaaaccagcgacacgccggtacaaaaaacgcgtcaggaaaaaacgtccgccgcgacgagcgccagcgtaacgttaagcgacgcgcaagcgaagctcatgcagccaggcgtcagcgacattaatatggaacgcgtcgaagcattaaaaacggctatccgtaacggtgagttaaaaatggatacgggaaaaatagcagactcgctcattcgcgaggcgcagagctacttacagagtaaaaaa

SEQ ID NO: 2 - DNA, 51 bp, synthetic construct
  source 1..51: mol_type = other DNA
ggcgaagaggaattacaggaaaaccaggaattgatccgtgaaaaatccaat

SEQ ID NO: 3 - DNA, 957 bp, synthetic construct
  source 1..957: mol_type = other DNA
aggaggtttgatcctatgggaacaatgcatacatccgagttgctaaaacacatttatgacatcaatttgtcatatttactccttgcacagcgtttgatcgtccaggacaaagcatctgcgatgttccgcctcggtatcaacgaagagatggcaaacacactgggcgcgttgaccctgccgcagatggtcaaactggcggagacgaaccagttagtttgtcatttccggtttgacgatcatcagacgatcacccgtttgactcaggattcgcgcgtcgatgacttacagcagattcacacaggtatcatgctttcaacgcgtctgctcaatgaagtggacgatacggcgcgtaagaaaagggcatgaaagggcatgataatgagtgaaaaaagcattgttcaggaagctcgcgatatccagttggcgatggagttgattaatcttggcgctcgtctacaaatgctggaaagcgaaacacagctcagccgtggtcgcctcatcaggctgtacaaagaattacgcggtagcccgccgcctaaagggatgctgccattttcgacagactggtttatgacctgggagcaaaatattcatgcctccatgttctgcaacgcctggcaatttttactgaagaccggcttatgcagcggtgtggatgcggtgattaaagcttatcggctttatcttgagcagtgtccgcaaccgcctgaagggccgttgttggcgctgactcgcgcatggacgctggtgcgttttgttgaaagtgggttgcttgaattgtcgagctgtaactgctgcggtgggaactttattacccatgcgcatcagcccgtaggcagctttgcgtgtagtttatgccagccgccatcccgcgcagtaaaaagacgtaaactttcccgagatgctgccgatattattccacaactgctggatgaacagatcgaacaggctgtttaa

SEQ ID NO: 4 - DNA, 18 bp, synthetic construct
  source 1..18: mol_type = other DNA
catcaccatcaccatcac

SEQ ID NO: 5 - DNA, 42 bp, synthetic construct
  source 1..42: mol_type = other DNA
ggcggcagcagccatcaccatcaccatcacagcagcggcggc

SEQ ID NO: 6 - DNA, 407 bp, synthetic construct
  source 1..407: mol_type = other DNA
gctagcaggaggtttgacctatgagcattgaccgtacctcacctttgaaacccgttagcactgtccagacgcgcgaaaccagcgacacgccggtacaaaaaacgcgtcaggaaaaaacgtccgccgcgacgagcgccagcgtaacgttaagcgacgcgcaagcgaagctcatgcagccaggcgtcagcgacattaatatggaacgcgtcgaagcattaaaaacggctatccgtaacggtgagttaaaaatggatacgggaaaaatagcagactcgctcattcgcgaggcgcagagctacttacagagtaaaaaacatcaccatcaccatcacagcagcggcggcatgggcgaagaggaattacaggaaaaccaggaattgatccgtgaaaaatccaattaagagctc

SEQ ID NO: 7 - DNA, 419 bp, synthetic construct
  source 1..419: mol_type = other DNA
gctagcaggaggtttgacctatgagcattgaccgtacctcacctttgaaacccgttagcactgtccagacgcgcgaaaccagcgacacgccggtacaaaaaacgcgtcaggaaaaaacgtccgccgcgacgagcgccagcgtaacgttaagcgacgcgcaagcgaagctcatgcagccaggcgtcagcgacattaatatggaacgcgtcgaagcattaaaaacggctatccgtaacggtgagttaaaaatggatacgggaaaaatagcagactcgctcattcgcgaggcgcagagctacttacagagtaaaaaaggcggcagcagccatcaccatcaccatcacagcagcggcggcatgggcgaagaggaattacaggaaaaccaggaattgatccgtgaaaaatccaattaagagctc

SEQ ID NO: 8 - DNA, 58 bp, synthetic construct
  source 1..58: mol_type = other DNA
gatcgatcgagctcaggaggtttgatcctatgggaacaatgcatacatccgagttgct

SEQ ID NO: 9 - DNA, 49 bp, synthetic construct
  source 1..49: mol_type = other DNA
gatcgatcgtcgacttaaacagcctgttcgatctgttcatccagcagtt

SEQ ID NO: 10 - DNA, 1107 bp, synthetic construct
  source 1..1107: mol_type = other DNA
atgaaaaatgttggttttatcggctggcgcggaatggtcggctctgttctcatgcaacgcatggtagaggagcgcgatttcgacgctattcgccctgttttcttttctacctcccagtttggacaggcggcgcccaccttcggcgacacctccaccggcacgctacaggacgcttttgatctggatgcgctaaaagcgctcgatatcatcgtgacctgccagggcggcgattataccaacgaaatttatccaaagctgcgcgaaagcggatggcagggttactggattgatgcggcttctacgctgcgcatgaaagatgatgccattattattctcgacccggtcaaccaggacgtgattaccgacggcctgaacaatggcgtgaagacctttgtgggcggtaactgtaccgttagcctgatgttgatgtcgctgggcggtctctttgcccataatctcgttgactgggtatccgtcgcgacctatcaggccgcctccggcggcggcgcgcgccatatgcgcgagctgttaacccagatgggtcagttgtatggccatgtcgccgatgaactggcgacgccgtcttccgcaattcttgatattgaacgcaaagttacggcattgacccgcagcggcgagctgccggttgataactttggcgtaccgctggcgggaagcctgatcccctggatcgacaaacagctcgataacggccagagccgcgaagagtggaaaggccaggcggaaaccaacaagattctcaatactgcctctgtgattccggttgatggtttgtgtgtgcgcgtcggcgcgctgcgctgtcacagccaggcgttcaccatcaagctgaaaaaagaggtatccattccgacggtggaagaactgctggcggcacataatccgtgggcgaaagtggtgccgaacgatcgtgatatcactatgcgcgaattaaccccggcggcggtgaccggcacgttgactacgccggttggtcgtctgcgtaagctgaacatggggccagagttcttgtcggcgtttaccgtaggcgaccagttgttatggggcgccgccgagccgctgcgtcgaatgctgcgccagttggcgtag